(19) United States
(12) Patent Application Publication
(10) Pub. No.: US 2021/0125717 A1
(43) Pub. Date: Apr. 29, 2021

(54) METHODS AND ANALYTICAL TOOLS FOR THE STUDY AND TREATMENT OF EPILEPTOGENESIS

(71) Applicant: University of Southern California, Los Angeles, CA (US)

(72) Inventors: Dominique Duncan, Los Angeles, CA (US); Arthur Toga, Los Angeles, CA (US)

(21) Appl. No.: 17/255,961

(22) PCT Filed: Jun. 10, 2019
(86) PCT No.: PCT/US19/36393

§ 371 Date: Dec. 23, 2020

Related U.S. Application Data
(60) Provisional application No. 62/690,292, filed on Jun. 26, 2018.

(51) Int. Cl. G16H 40/63; G16H 30/20; G16H 20/30; G06N 20/00

(52) U.S. Cl. CPC G16H 40/63; G06N 20/00; G16H 20/30; G16H 30/20

(57) ABSTRACT

Methods, systems, and apparatus for identifying biomarkers of epileptogenesis. The repository and analytics system includes multiple data source devices. The multiple data source devices are configured to provide neurological data. The repository and analytics system includes a repository and analytics platform that is coupled to the multiple source devices. The repository and analytics platform is configured to determine a relationship or pattern within the neurological data based on a linked set of neurological data. The repository and analytics platform is configured generate a visualization that identifies biomarkers of epileptogenesis based on the relationship or pattern. The repository and analytics system includes a client device. The client device is configured to display the visualization.

### CROSS-REFERENCE TO RELATED APPLICATIONS

This application claims priority to and the benefit of U.S. Provisional Patent Application No. 62/690,292 titled “METHODS AND ANALYTICAL TOOLS FOR THE STUDY AND TREATMENT OF EPILEPTOGENESIS,” filed on Jun. 26, 2018, and the entirety of which is hereby incorporated by reference herein.

### STATEMENT REGARDING GOVERNMENT RIGHTS

This invention was made with Government support under Award Numbers U54NS100064 (EpiBioS4Rx), NIH P41-EB015922 and NIH U54-EB020406 awarded by the National Institute of Neurological Disorders and Stroke (NINDS) of the National Institutes of Health (NIH). The Government has certain rights in this invention.

### BACKGROUND

### 1. Field of the Invention

This specification relates to the study and treatment of epileptogenesis.

### 2. Description of the Related Art

There have been efforts to create centralized data archives, but it has proven to be especially challenging for human neurophysiological data for many reasons, such as large file sizes, varying formats, privacy constraints, and funding. Two examples of centralized EEG databases that have been developed include Epilepsiae and IEEG.ORG. Epilepsiae stores recordings from 275 individuals with epilepsy, with a total recording time of more than 40,000 hours. Investigators can export the data locally for analysis. IEEG.ORG hosts academic and clinical datasets of scalp and intracranial EEG, just over 800 of which are shared publicly, from both animal models of epilepsy and patients. This platform uses Amazon cloud services. Access for Epilepsiae is restricted to scientific groups that financially contribute to the maintenance of the database, which has resulted in fewer people using the platform. IEEG.ORG is free and accessible to the epilepsy research community.

The number of large databases and related neurological disease-focused consortia around the world has grown rapidly in recent years, which demonstrates the importance of transparency in large-scale projects and the sharing of data that are collected. Larger datasets from preclinical studies are now emerging. Beyond sharing data, to encourage the most impactful outside collaborations and scientific discoveries, the data must be well organized and annotated (i.e., for EEG). Furthermore, the data sharing platform must be user friendly and straightforward to use.

It would be desirable, therefore, to overcome these and other deficiencies of existing systems and methods with new and improved approaches. More specifically, systems and methods that have the ability to store and share disparate types of data, including imaging, electrophysiology, and clinical data, from both humans and animals, on one platform that includes not only options for data visualization but also a wide variety of analytic tools that are integrated across different programming languages are needed.

### SUMMARY

In general, one aspect of the subject matter described in this specification is embodied in a device, system and/or apparatus for the study and treatment of epileptogenesis. The repository and analytics system includes multiple data source devices. The multiple data source devices are configured to provide neurological data. The repository and analytics system includes a repository and analytics platform that is coupled to the multiple source devices. The repository and analytics platform is configured to determine a relationship or pattern within the neurological data based on a linked set of neurological data. The repository and analytics platform is configured generate a visualization that identifies biomarkers of epileptogenesis based on the relationship or pattern. The repository and analytics system includes a client device. The client device is configured to display the visualization.

These and other embodiments may optionally include one or more of the following features. The multiple data sources devices may include a first data source device and a second data source device. The first data source device may be configured to provide a first set of neurological data. The second data source device may be configured to provide a second set of neurological data. The neurological data may include the first set of neurological data and the second set of neurological data.

The multiple data source devices may include a third data source device. The third data source device may be configured to provide a third set of neurological data. The first set of neurological data may be collected from a first subject. The second set of neurological data may be collected from a second subject. The first set of neurological data may be in a first format. The second set of neurological data may be in a second format. The repository and analytics platform may be configured to standardize or convert the first set of neurological data and the second set of neurological data into a standard format.

The repository and analytics platform may be configured to determine a shared attribute between a subset of the first set of neurological data and a subset of the second set of neurological data. The repository and analytics platform may be configured to combine or link the subset of the first set of neurological data with the subset of the second set of neurological data to form the linked set of neurological data based on the shared attribute.

The neurological data may include multi-modal data including neuroimaging, electrophysiology, molecular sample data, serological sample data or tissue sample data. The repository and analytics platform may be configured to identify multiple biomarkers indicating epileptogenesis. The repository and analytics platform may be configured to de-identify the neurological data, which may include removing any personal identifiable information. The repository and analytics platform may be configured to assign a global unique identifier to the neurological data information and validate a quality of the neurological data.

In another aspect, the subject matter is embodied in a repository and analytics platform. The repository and analytics platform includes a memory. The repository and analytics platform includes one or more processors coupled to the memory and configured to execute instructions store in the memory. The one or more processors perform operations including obtaining, from multiple data source devices, neurological data including a first set of neurological data and a second set of neurological data. The operations include combining or linking a subset of the first set of neurological data with a subset of the second set of neurological data to form a linked set of neurological data. The operations include determining a relationship or pattern within the neurological data based on the linked set of neurological data. The operations include displaying, on a client device, a visualization that identifies biomarkers of epileptogenesis based on the relationship or pattern.

In another aspect, the subject matter is embodied in a method for identifying biomarkers. The method includes obtaining, from multiple data source devices and using a processor, neurological data including a first set of neurological data and a second set of neurological data. The method includes combining or linking, using the processor, a subset of the first set of neurological data with a subset of the second set of neurological data to form a linked set of neurological data. The method includes determining, using the processor, a relationship or pattern within the neurological data based on the linked set of neurological data. The method includes displaying, on a client device and using the processor, a visualization that identifies biomarkers of epileptogenesis based on the relationship or pattern.

### BRIEF DESCRIPTION OF THE DRAWINGS

Other systems, methods, features, and advantages of the present invention will be apparent to one skilled in the art upon examination of the following figures and detailed description. Component parts shown in the drawings are not necessarily to scale, and may be exaggerated to better illustrate the important features of the present disclosure.

FIG. 1 shows a block diagram of a repository and analytics system according to an aspect of the invention.

FIG. 2 shows a diagram of example modules within the repository and analytics system of FIG. 1 according to an aspect of the invention.

FIG. 3 is a flow diagram of an example process for collecting and providing the neurological data for data archiving and biomarker identification using the repository and analytics system of FIG. 1 according to an aspect of the invention.

FIG. 4 is a flow diagram of an example process for processing and analyzing the collected neurological data using the repository and analytics system of FIG. 1 according to an aspect of the invention.

FIG. 5 shows a diagram that summarizes the collection and processing of the neurological data using the repository and analytics system of FIG. 1 according to an aspect of the invention.

FIGS. 6A-6C shows the results of analysis performed using the repository and analytics system of FIG. 1 according to an aspect of the invention.

### DETAILED DESCRIPTION

Disclosed herein are systems, devices and methods for the infrastructure and functionality of a centralized preclinical and clinical data repository and analytics platform (“repository and analytics system”) to support importing heterogenous multi-modal data. The repository and analytics system automatically and manually links data across multiple modalities, sites and searching content. The repository and analytics system applies innovative image and electrophysiology identifies candidate biomarkers from magnetic resonance imaging (MRI), electroencephalogram (EEG) and multi-modal data to track the probability of developing epilepsy over time. This allows for the study of epileptogenesis after a traumatic brain injury.

Moreover, a fundamental challenge in discovering biomarkers that may indicate epileptogenesis, after a traumatic brain injury (TBI), is that the process is multifactorial and crosses multiple modalities. Rather than considering only one type of data, the repository and analytics system collects and analyzes multi-modal data, including neuroimaging, electrophysiology, and molecular/serological/tissue. Furthermore, the repository and analytics system facilitates analysis and collaboration among scientists from various centers around the world. The repository and analytics system uses innovative analytic tools that are shared with the broader epilepsy research community so that others may use the tools in addition to their own tools to advance research in this field in general, in addition to identifying biomarkers of epileptogenesis after TBI.

Additionally, investigators must have access to a large number of high quality, well-curated data points and study subjects in order for biomarker signals to be detectable above the noise inherent in complex phenomena, such as epileptogenesis, TBI, and conditions of data collection. Since data generating and collecting sites are spread worldwide among different laboratories, clinical sites, heterogeneous data types, and formats, and across multi-center preclinical trials, there is a need for a central repository of the data collection. The repository and analytics system standardizes the data and provides tools for searching, viewing, annotating, and analyzing the data. By centralizing an enduring data archive, biobank, and analytic tools, researchers may identify and validate biomarkers of epileptogenesis in studies using various types of data.

Beyond creating a centralized data repository, the repository and analytics system has innovative standardization/co-registration references, fully supported by novel image and electrophysiology processing methods to extract candidate biomarkers from the diverse data. Not only does a well-curated and standardized multi-modal dataset facilitate the development of models of epileptogenesis, but it also ensures that such models are statistically significant and can be validated. Thus, the repository and analytics system advantageously provides a platform that stores and shares disparate types of data, including imaging, electrophysiology, and clinical data, from both humans and animals, on a single platform that includes not only options for data visualization but also a wide variety of analytic tools that are integrated across multiple programming languages.

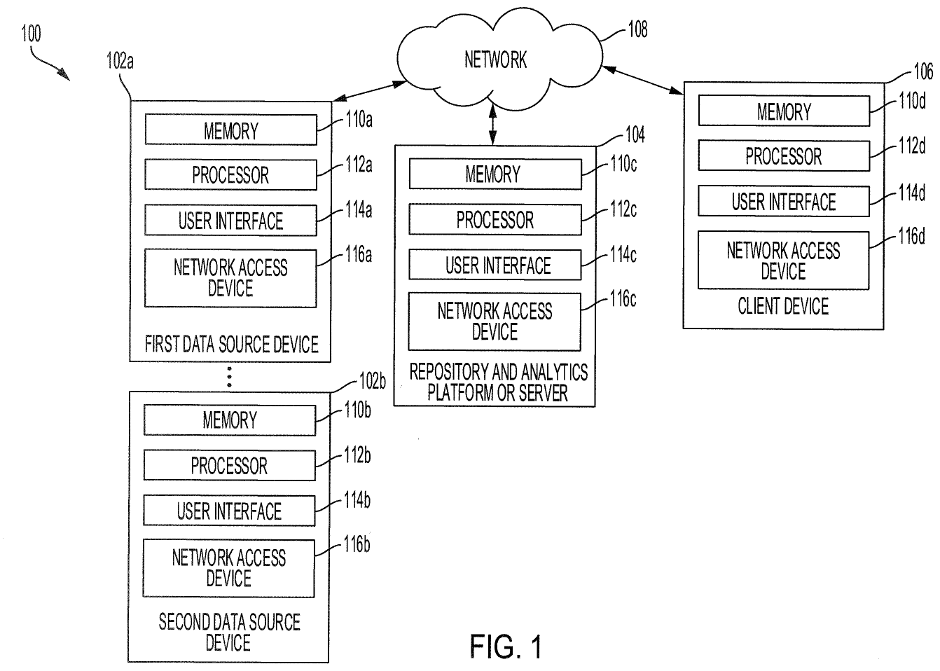

FIG. 1 shows a block diagram of a repository and analytics system 100. The repository and analytics system 100 includes one or more data source devices 102a-b, a repository and analytics platform 104 and a client device 106. The repository and analytics system 100 may have a network 108 that links or couples the one or more data source devices 102a-b, the repository and analytics platform 104 and/or the client device 106. The network 108 may be a local area network (LAN), a wide area network (WAN), a cellular network, the Internet, other wired or wireless communication, or combination thereof, that connects, couples and/or otherwise communicates between the various components of the repository and analytics system 100, such as the one or more data source devices 102, the repository and analytics platform 104 and/or the client device 106.

The one or more data source devices 102a-b may include multiple data source devices 102a-b, such as a first data source device 102a and/or a second data source device 102b. A data source device 102a-b is a device that obtains neurological data of a subject or patient, either human or animal, and may provide the neurological data to the repository and analytics platform 104. For example, the data source device 102a-b may be an electroencephalogram (EEG) scanner, a magnetic resonance imager (MRI), or a diffusion tensor imager (DTI) and the neurological data may be collected as a result of a clinical or behavioral study of a person or an animal.

Each data source device 102a-b may obtain and provide a set of neurological data that is formatted in a specific format based on the type or kind of data source device 102a-b. For example, the MRI may obtain and provide a set of neurological data in one format, whereas the DTI may obtain and provide another set of neurological data in another different format. The different data source devices 102a-b may obtain the neurological data by measuring, scanning, testing or otherwise interacting with the subject. In some implementations, a user may enter the neurological data into the data source device 102a-b.

The neurological data may include multiple data points collected at different points in time. Each data point may be a test sample of the subject using one or more of the data source devices 102a-b at a particular point in time. Multiple data points may be sampled of multiple subjects including animals and/or humans over the same or different periods of time using multiple data source devices 102a-b to accumulate and generate the neurological data used to analyze to identify biomarkers.

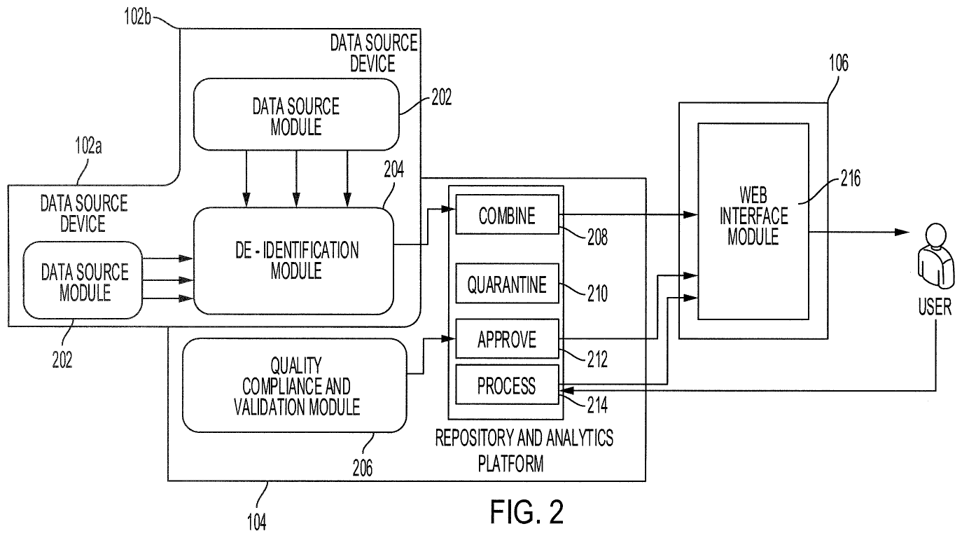

The one or more data source devices 102a-b may include a memory 110a-b, one or more processors 112a-b, a user interface 114a-b and/or a network access device 116a-b. The memory 110a-b may store instructions that are executed by the one or more processors 112a-b. The memory 110a-b may store the raw neurological data. Raw neurological data may be neurological data obtained by the one or more data source devices that is in the original format and not in a standardized format that has been standardized by the repository and analytics platform 104 for processing and analysis. One or more processors 112a-b are coupled to the memory 110a-b. The one or more processors 112a-b may operate a data source module 202, as shown in FIG. 2 for example, that collects the raw neurological data of the subject and provides the raw neurological data to the repository and analytics platform 104. The one or more processors 112a-b may operate a de-identification module 204. The de-identification module 204 removes the personal identifiable information including the first and last names of the subject from the neurological data to ensure anonymity of the subject prior to sending the neurological data to the repository and analytics platform 104. Then, the de-identification module 204 may assign one or more global unique identifiers (GUIDs) to the neurological data to ensure that the neurological data from the same subject is identified and is not used or counted multiple times within the neurological data. The GUIDs distinguish the subjects uniquely across datasets of the neurological data collected from various data source devices 102a-b. By using GUIDs, the neurological data from the subject may remain anonymous while also providing a way to identify the data. The de-identification module 204 allows for cross-comparisons of GUIDs between different datasets and different devices without revealing the internal has codes used for subject identification.

A network access device 116a-b may be coupled to the one or more processors 112a-b and may transmit or otherwise provide the raw neurological data across the network 108 to the repository and analytics platform 104. In some implementations, the user interface 114a-b may be used to receive and/or obtain raw neurological data on the one or more data source devices 102a-b. For example, a user, such as a doctor, may enter the raw neurological data into the user interface 114a-b.

The repository and analytics platform 104 may be a server and may be coupled to one or more data source devices 102a-b via the network 108. The repository and analytics platform 104 may have a network access device 116c that receives the raw neurological data from the one or more data source devices 102a. The repository and analytics platform 104 may include a memory 110c, one or more processors 112c and a user interface 114c. The one or more processors 112c may be coupled with the user interface 114c, which allows for a user to configure the repository and analytics platform 104. The memory 110c may store the raw neurological data and/or neurological data that has been processed, de-identified, validated or otherwise approved. The memory 110c may store the instructions to perform the approval process. The one or more processors 112c may be coupled to the memory 110c and execute the instructions store in the memory to perform the approval process of the neurological data along with generating visualizations to assist a user of the client device 106 to identify candidate biomarkers of epileptogenesis. The one or more processors 112 may operate a quality compliance and validation module 206. The quality compliance and validation module 206 automatically detects new data, validates the data, maps the data to a common data model (where applicable), and pre-indexes the clinical data by features and values to aid in search and co-registration. Through a federated architecture, key components of the data may be distributed while quality control and provenance information is maintained with all neurological data. Moreover, the multi-modal data may be checked for quality and reviewed. The quality compliance and validation module 206 normalizes and harmonizes signal, image and other data and allows automated pre-processing that generates vector statistics and derived images to assess data quality. The neurological data may be run through automated artifact detection algorithms in preparation for initial biomarker processing.

The one or more processors 112c may have other modules, such as the combine module 208 that identifies shared attributes and links different datasets based on the shared attribute to identify relationships and patterns within the datasets, a quarantine module 210 that performs the quality control and validation of the neurological data so that the neurological data is validated, approved or otherwise is identified as quality data, an approve module 212 that approves neurological data of a particular quality that is sufficient for analysis and presentation and/or a process module 214 that performs analysis on the neurological data and other functions such as search and retrieval of the neurological data. The repository and analytics platform 104 may interact with the client device 106 to perform the search, retrieval and visualization of the neurological data and communicate via the network access device 116c.

The repository and analytics system 100 includes a client device 106. The client device 106 may be a computing device, such as a personal computing device, smartphone, laptop, or tablet, that has a user interface, such as a web-based client interface, to receive user input, such as search queries, and display or provide results, such as visualizations that identify biomarkers, or otherwise present the neurological data. The client device 106 includes a memory 110d, one or more processors 112d, a user interface 114d and/or a network access device 116d. The client device 106 may be coupled to the repository and analytics platform 104 via the network access device 116d through the network 108. The memory 110d may store instructions that the one or more processors 112d coupled to the memory 110d execute to run an application, such as a web-based client, to perform search queries and display visualizations to identify the candidate biomarkers.

The one or more processors 112d may operate a web interface module 216. The web interface module 216 allows for user-friendly data search and navigation of the neurological data. The web interface module may be a web-client. The user interface 114d may receive user input that includes the search queries and may display the visualizations on a display via the web-client. The search may be performed across the neurological data that is interlinked and co-registered across datasets and modalities. The search may find interlinked combinations of data that match the desired criteria. This enables sophisticated custom searches that match the functionality of predefined query forms. Users can browse data in a visual representation and pivot from one data view or modality to another. The web interface module may enforce access control and sharing mechanisms, such as permission management.

The one or more processors 112a-d may each be implemented as a single processor or as multiple processors. The one or more processors 112a-d may be electrically coupled to, connected to or otherwise in communication with the corresponding memory 110a-d and/or network access device 116a-d and/or user interface 114a-d on the respective device, such as the data source devices 102a-b, the repository and analytics platform 104 and/or the one or more client devices 106.

The one or more memories 110a-d may be coupled to the one or more processors 112a-d and store instructions that the processors 112a-d execute. The one or more memories 110a-d may include one or more of a Random Access Memory (RAM) or other volatile or non-volatile memory. The one or more memories 110a-d may be a non-transitory memory or a data storage device, such as a hard disk drive, a solid-state disk drive, a hybrid disk drive, or other appropriate data storage, and may further store machine-readable instructions, which may be loaded and executed by the one or more processor 112a-d. Moreover, the one or more memories 110a-d may be used to store one or more applications, such as a web-based client.

The one or more user interfaces 114a-d may include any device capable of receiving user input, such as a button, a dial, a microphone, or a touch screen, and any device capable of output, e.g., a display, a speaker, or a refreshable braille display. The one or more user interfaces 114a-d allow a user to communicate with the one or more processors 112a-d, respectively, and/or display information, such as a visualization or search results.

The one or more network access devices 116a-d may include a communication port or channel, such as one or more of a Wi-Fi unit, a Bluetooth® unit, a radio frequency identification (RFID) tag or reader, or a cellular network unit for accessing a cellular network (such as 3G, 4G or 5G). The one or more network access device 116a-d may transmit data to and receive data among the components of the repository and analytics system 100.

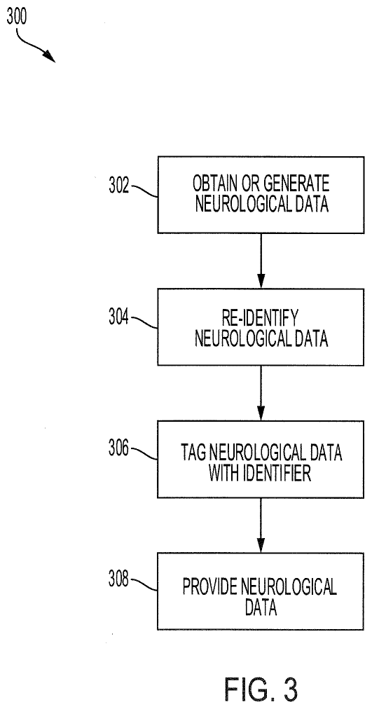

FIG. 3 is a flow diagram of an example process 300 for collecting and providing the neurological data for data archiving and biomarker identification. One or more computers or one or more data processing apparatuses, for example, the processors 112a-d of the repository and analytics system 100 of FIG. 1, and in particular the one or more processors 112a-b of the one or more data source devices 102a-b, appropriately programmed, may implement the process 300.

The repository and analytics system 100 may obtain or generate neurological data (302). The neurological data may include multi-modal data collected using one or more data source devices 102a-b. The multi-modal data may include data from neuroimaging, electrophysiology, molecular samples, serological samples, and tissue samples from an animal, person or other subject. Other data may include ICU physiological data, demographic information, outcome measures and prospective research data. One or more data source devices 102a-b that collect the neurological data may include an electroencephalogram (EEG) scanner, a magnetic resonance imager (MRI), or a diffusion tensor imager (DTI) and the neurological data may be collected as a result of a clinical or behavioral study of a person or an animal. The one or more data source devices 102a-b may be connected, coupled to or an include a sensor that detects, measures or collects the data. In some implementations, the one or more data source devices 102a-b may receive user input that includes the neurological data. The neurological data may be in a raw format that is specific to the data source device 102a-b, which may need to be converted for further processing.

The repository and analytics system 100 may de-identify neurological data or otherwise remove personal identifiable information associated with the neurological data (304). The one or more data source devices 102a-b remove the first names, the last names and other personal identifiable information that is associated with the subject. This ensures anonymity of the subject prior to sending the neurological data to the repository and analytics platform 104. The one or more data source devices 102 may remove the personal identifiable information prior to providing the neurological data to the repository and analytics platform 104, which ensures that when the data is collected and aggregated with other neurological data from other data source devices 102a-b the personal identifiable information has been removed and is not attached with the analyzed data.

Once the personal identifiable information is removed, the repository and analytics system 100 associates, attaches or otherwise tags the neurological data with a global identifier (306). The one or more data source devices 102a-b and/or the repository and analytics platform 104 may assign a global identifier, such as one or more GUIDs to the neurological data to ensure that the neurological data from the same subject is identified and is not used or counted multiple times within the neurological data.

Within the repository and analytics system, the one or more data sources devices 102a-b may provide the neurological data to the repository and analytics platform 104 (308). Different types of neurological data may be uploaded to the repository and analytics platform 104 including EEG, which uses the European Data Format (EDF)+ format. The repository and analytics platform 104 may receive different sets of neurological data from each of the one or more data source devices 102a-b. The different sets of neurological data may have different formats and may be tagged with a format identifier. For example, data collected by an MRI scanner may be in a different format than data collected by an EEG scanner or a CT scanner. Other formats may include DICOM, ECAT, HRRT and EDF.

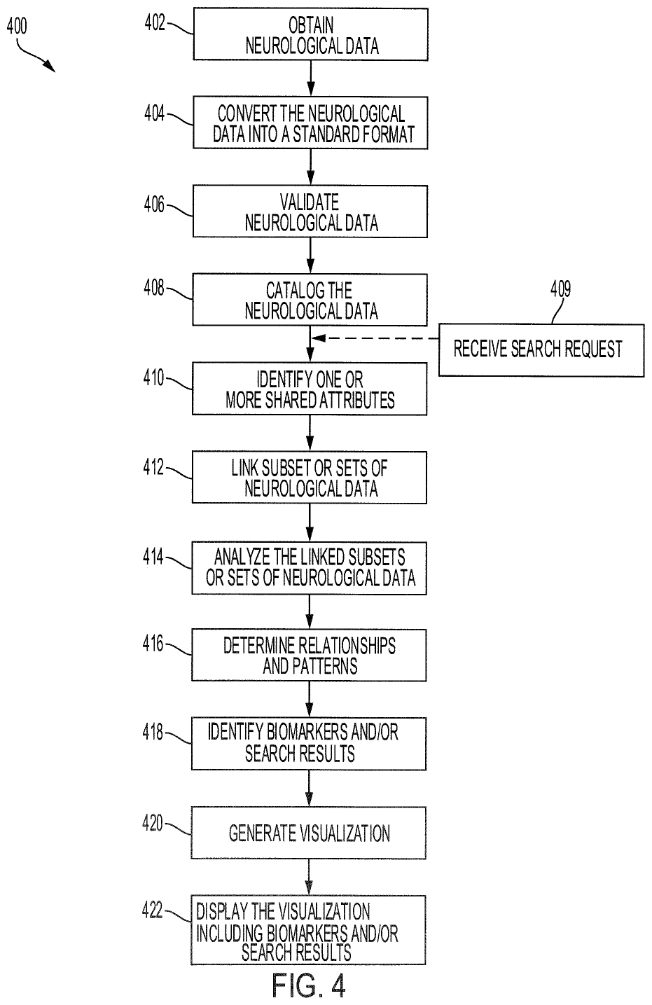

FIG. 4 is a flow diagram of an example process 400 for processing and analyzing the collected neurological data. One or more computers or one or more data processing apparatuses, for example, the processors 112a-d of the repository and analytics system 100 of FIG. 1, and in particular the one or more processors 112c of the repository and analytics platform 104, appropriately programmed, may implement the process 400.

The repository and analytics system 100 may receive or otherwise obtain the neurological data from the one or more data source devices 102a-b (402). The neurological data may be in different formats and may be obtained over the network 108 using the network access device 116c.

The repository and analytics system 100 may convert the neurological data that is received in the different formats from the different data source devices 102a-b into a standard format (404). The repository and analytics system 100 may read a tag or parse the neurological data to determine the existing format and apply an algorithm to convert the existing format to the standard format. By automating the process, the neurological data may be shared across different platforms, which reduces coordination challenges.

Once converted, the repository and analytics system 100 may validate and perform quality control on the neurological data (406). The repository and analytics platform 104 may measure the signal to noise ratio of the neurological data and filter the noise out to below a threshold amount to achieve the approved neurological data. The repository and analytics platform 104 may determine which data points of the neurological data are reliable and which are unreliable. The data points that fall within a preselected or calculated data range may be considered reliable, whereas, the data points that fall outside the preselected or calculated data range may be considered unreliable. In some implementations, the repository and analytics platform 104 generates vector statistics and derived images to assess the data quality.

The repository and analytics system 100 catalogs the neurological data (408). The repository and analytics platform 104 may receive the neurological data and identifies and extracts a subset of metadata attributes, which the repository and analytics platform 104 uses to catalog and describe the neurological data to support database searches. The check-in is typically completed within 3 minutes, at which time the neurological data becomes immediately available.

The repository and analytics system 100 may receive a search query or request (“search request”) (409). The repository and analytics system 100 may receive the search request from one or more client devices 106. The search request may be a request to identify neurological data that matches a particular set of criteria or filters, such as species, keywords, demographic or individual subject information including age, weight, race, gender, illness or location of biomarkers, and/or type of neurological data including biosamples, image data or EEG data. The results of the search request are presented to the client device 106 after analysis of the neurological data.

Once checked-in, the repository and analytics system 100 may identify shared attributes between the different sets of the neurological data (410). The shared attribute may be shared between subsets or portions of the different sets of neurological data. For example, the shared attribute may be a common patient or subject that is included in two or more sets of neurological data that each are obtained from different data source devices 102a-b.

A shared attribute is a commonality, common factor or other common criteria that is present, related or otherwise associated between two different sets of neurological data. For example, an MRI scan and a CT scan may have been taken at the same time for the same patient, and thus, share the same timeframe of the condition of the same subject. In another example, an EEG scan of animal with an illness and an EEG scan of human with the same illness may share the shared attribute of the disease. Other shared attributes may include an injury location, such as a similar or same region of the brain, severity of the injury, height of subject, weight of subject, race, gender, age or other characteristic of the subject, a common disease, stage or progression of the disease, timeframe or prescribed treatment to the subject or a combination thereof.

The repository and analytics system 100 may link different subsets or sets of neurological data based on the shared attribute (412). The repository and analytics platform 104 associates indexes between the subsets or sets of neurological data to link the different subsets or sets of neurological data. Once a shared attribute between two different subsets or sets of neurological data is identified, the repository and analytics system 100 may link the two different subsets. The link may indicate that there is a similarity or relationship that exists between the two different subsets or sets of neurological data. When a user identifies, requests or otherwise searches for criteria related to a first subset or set of neurological data, the link allows the repository and analytics platform 104 to identify other linked subsets or sets of neurological data that may be interrelated or otherwise associated with the first subset or set of neurological, which may be of value or pertinent to the user in response to the search criteria. By linking the two different subsets or sets of neurological data, the repository and analytics platform 104 may follow the development of epilepsy as these two different subsets or sets of neurological data progress over time to determine relationships and/or patterns associated with the different subsets or sets of neurological data.

Moreover, the repository and analytics system 100 may receive user input that identifies links between different subsets or sets of neurological data. For example, a user can look at an EEG and find corresponding patient's imaging data to see spatially from where the EEG recordings were taken. Moreover, a user is able to compare clinical data over various time points with the different subsets or sets of data, such as from the EEG and MRI data. Thus, the user may provide and the repository and analytics system 100 may obtain the linkage across different data modalities to identify relationships and patterns and perform further analysis including dimensionality reduction and pattern recognition of identifying epileptogenesis after TBI.

The repository and analytics system 100 analyzes the linked sets of neurological data (414) and determines relationships and patterns based on the linked sets of neurological data (416). The repository and analytics platform 104 analyzes the linked sets of neurological data and determines the relationships and patterns to identify candidate biomarkers of epileptogenesis. The relationships and patterns may include similarities or differences between the different subsets of neurological data over periods of time.

The repository and analytics platform 104 may use different algorithms and/or processes to analyze the different types of neurological data. In order to identify key features within the sets of neurological data the repository and analytics system 100 may perform dimensionality reduction, split the neurological data into different submatrices, reshape the neurological data into vectors, compute histograms, compute between consecutive, random shuffle, and random projection, calculate covariance matrices, compute eigenvalue decomposition, and calculate inverse matrices.

For example, the repository and analytics platform 104 may develop a unified coordinate space for seizure onset locations across various brains, including animal and human MRI, using string similarity and value overlap to predict that different contributor metadata fields are the same, and providing graphical interfaces for linking data. Additionally, since MRI data includes structural, functional (resting state) and diffusion weighted measures, analysis of the MRI data may include structural analyses to measure each subject's intracranial volumes as well as gray matter volumes and other anatomical measures. The analysis of the MRI data may also include using statistical parametric mapping to ascertain brain activation in different regions. Additionally, functional connectivity analysis may be performed to examine network connectivity in comparison to non-TBI data, to determine abnormally active or inactive networks. Lastly, the diffusion weighted analyses may include constructing each subject's fractional anisotropy (FA) maps in addition to measuring each patient's apparent diffusion coefficient to assess white matter (WM) integrity and connectivity. These FA maps of TBI data may be compared to five normal, non-TBI data in group analysis via tract based spatial statistics.

The repository and analytics platform 104 may compare human and animal neuroimaging data. In particular, the repository and analytics system 100 may compare the characteristics and integrity of human and animal neuroimaging data to examine tract variabilities.

In another example, the repository and analytics platform 104 may use DTI data to determine connectivity between all pairs of gyral and sulcal structures in the presence of brain trauma. The connectivity between all brain regions is calculated from DTI volumes acquired longitudinally from each subject. The repository and analytics system 100 may use diffusion tractography to determine connectivity properties, such as connectivity density, WM bundle length, and FA, and each subject's weighted connectivity matrix. The connectivity may be assessed systematically within each subject using purpose-built workflows for multi-modal co-registration of MRI data. This is followed by calculation of (i) inter-regional connectivity matrices and (ii) longitudinal changes in connectivity topology using network-theoretic descriptors of nodal and network-wide segregation (clustering coefficient, modularity, etc.) and integration (characteristic path length, global efficiency, etc.). Additional network-theoretic measures (scale freedom, small worldness, robustness, centrality, degree distribution and communication efficiency) may be calculated.

In another example, the repository and analytics platform 104 may perform analysis of EEG data including export of artifact reduced waveform data, seizure and spike detection, wavelets, matching pursuit, correlation, Fast Fourier Transform (FFT) phase, period evolution, and other EEG analysis. Due to the sheer volume of EEG data, the repository and analytics system may apply a variety of dimensionality reduction techniques to the EEG data for both preclinical and clinical data. Also, to increase the ease of understanding the high dimensional data and outline trends, the data may be reduced to lie on a nonlinear manifold of lower, intrinsic dimensionality to remove excessive noise in the data.

Other analysis, such as Principal Component Analysis (PCA), Diffusion Maps, Laplacian Eigenmaps, Kernel PCA, and Unsupervised Diffusion Component Analysis (UDCA) may be used on the data of the subject. PCA is a linear dimensionality reduction method used by rotating data in a different orientation in the dimensional space by exposing the maximum variance. This detects and eliminates noise and collects the redundancy of the data. Kernel PCA is an extension of PCA that uses techniques of kernel methods. Laplacian Eigenmaps is a nonlinear dimensionality reduction method that assumes that data lies in a low dimensional manifold within the high dimensional space to produce a low dimensional dataset by preserving local properties of the manifold and minimizing the distance between a data point and its neighbor. Diffusion mapping is another nonlinear dimensionality reduction method where a family of embeddings of a dataset is computed into a low-dimensional Euclidean space whose coordinates can be computed from the eigenvectors and corresponding eigenvalues of a diffusion operator on the data. UDCA is an extension and adaptation of diffusion maps. Coordinates are constructed that generate efficient geometric representations of the complex data and noise is removed to extract the underlying brain activity that may be associated with biomarkers of epileptogenesis from the EEG data.

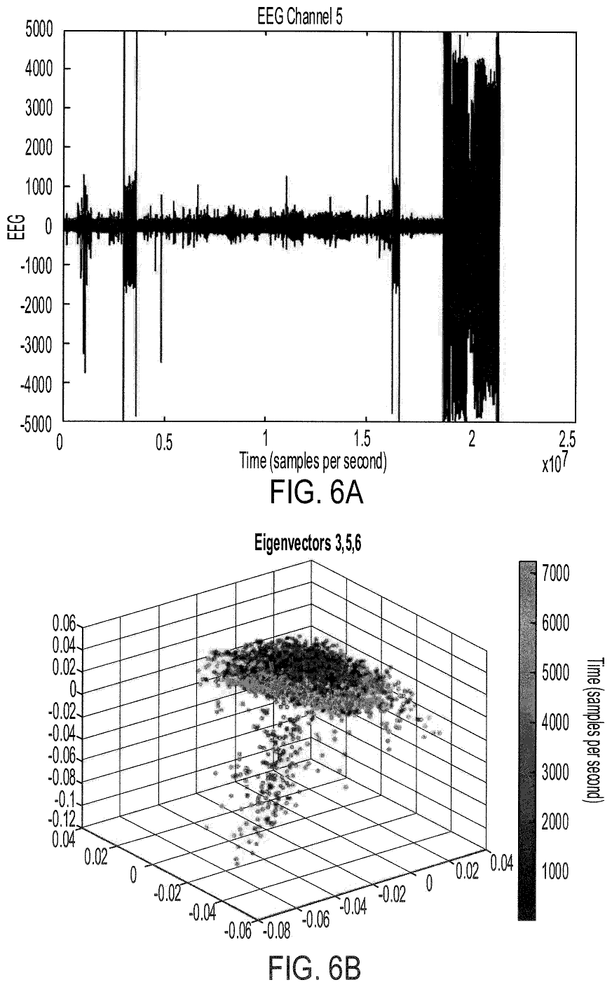

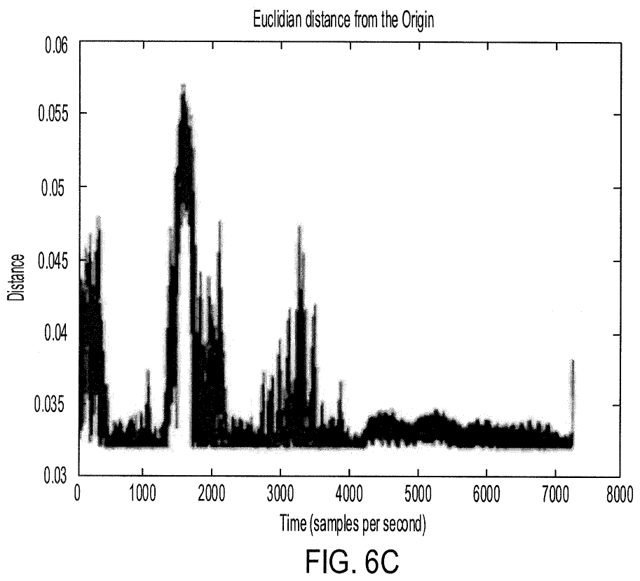

FIGS. 6A-6C show the results of when UDCA is applied to a sample of pre-ictal data. The repository and analytics platform 104 applies UDCA and separates pre-seizure features that are not apparent from visually inspecting the raw neurological data, and then plots the Euclidean distances of the points from the embedding to the origin to demonstrate setting a threshold of a chosen amplitude that can be used to automatically extract features of epileptogenesis after TBI. This reduces the noisy complex data, such as EEG and allows users to extract the underlying brain activity that may be associated with biomarkers of epileptogenesis. FIG. 6A plots the EEG data over a period of time. FIG. 6B plots the eigenvectors across the period of time and FIG. 6C plots the Euclidean distance from the origin over time.

The repository and analytics platform 104 may use artificial intelligence, such as machine algorithms, to train or model behavior of relationships and patterns to facilitate the analysis. For example, the repository and analytics platform 104 may identify the MRI data within the neurological data and segment the MRI data, e.g., separate the brain tissue from the non-brain tissue in the MRI data to outline the different brain regions within the MRI image using machine algorithms to automate the process.

The repository and analytics system 100 may identify biomarkers of epileptogenesis and/or search results based on the relationship or pattern (418) and generate a visualization that shows the biomarkers and/or visualizes the analysis performed over time and/or present one or more search results (420). The repository and analytics platform 104 may receive user input that includes one or more biomarkers and include the one or more biomarkers in the visualization to present and display to the user. The visualizations may use connectograms to present the visualizations. Moreover, the repository and analytics platform 104 may determine the search results, which are the indexed subsets or sets of neurological data that match the criteria or filters provided in the search request. Additionally, the repository and analytics platform 104 may provide the linked subsets or sets of neurological data that are associated and/or related, e.g., have a shared attribute, to the search results of the search request. By identifying the biomarkers and presenting the visualization to user, the user may assess the likelihood of the development of epilepsy and/or identify pre-cursors to the formation of epilepsy to assist in treatment.

The repository and analytics system 100 displays the visualization that includes the one or more biomarkers and/or the one or more search results (422). The repository and analytics platform 104 may transmit the visualization to the client device 106, which displays the visualization on the user interface, for example.

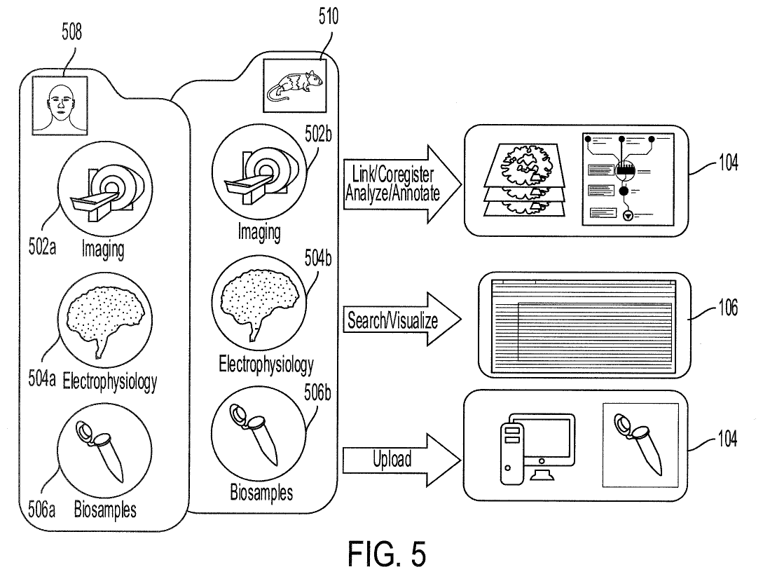

FIG. 5 shows a diagram that summarizes the collection and processing of the neurological data 502a-b, 504a-b, 506a-b using the repository and analytics system 100 of FIG. 1. One or more computers or one or more data processing apparatuses, for example, the processors 112a-d of the repository and analytics system 100 of FIG. 1, appropriately programmed, may implement the process 500.

One or more data source devices 102a-b may obtain or generate the neurological data 502a-b, 504a-b, 506a-b from a subject. The subject may be a human 508 or an animal 510. For example, the one or more data source devices 102a-b may include a magnetic resonance imager or diffusion tensor imager, which produces imaging data 502a for a human 508 and/or imaging data 502b for an animal 510, or an electrophysiology scanner, which produces electrophysiology data 504a for the human 508 and/or electrophysiology data 504b for the animal 510. In another example, the one or more data source devices 102a-b may include a device that captures or generates neurological data from biosamples, which may include molecular samples, serological samples, or tissue samples. The device that generates the neurological data from biosamples may provide biosample data 506a for the human 508 and/or biosample data 506b for the animal 510. The one or more data source devices 102a-b may perform de-identification and upload the neurological data 502a-b, 504a-b, 506a-b to the repository and analytics platform 104.

The repository and analytics platform 104 may organize and/or classify the different types of neurological data 502a-b, 504a-b, 506a-b data taken from the different subjects 508, 510 according to the type of subject they are taken from or other shared attribute. In some implementations, the one or more data source devices 102a-b may tag, organize or otherwise classify the different types of neurological data 502a-b, 504a-b, 506a-b prior to uploading the neurological data 502a-b, 504a-b, 506a-b to the repository and analytics platform 104.

After the neurological data has been uploaded, the repository and analytics platform 104 analyzes the neurological data 502a-b, 504a-b, 506a-b and identifies biomarkers of epileptogenesis. The repository and analytics platform 104 processes the analysis and generates a visualization that shows the biomarkers of epileptogenesis and/or other neurological data requested by a client device 106. Moreover, the repository and analytics platform 104 may receive or obtain search queries or requests of the collected neurological data from a web-based interface or client on the client device 106. The search queries or requests may include various parameters or filters to search. The filters or search criteria may include the type or species of the subject, such as animal or human, age ranges, weight ranges, height ranges or other characteristics of demographic information about the subject, for example. The visualization and/or search results are then displayed on the client device 106.

Exemplary embodiments of the systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A repository and analytics system, comprising:
a plurality of data source devices configured to provide neurological data;
a repository and analytics platform coupled to the plurality of data source devices and configured to:
determine a relationship or pattern within the neurological data based on a linked set of neurological data, and
generate a visualization that identifies biomarkers of epileptogenesis based on the relationship or pattern; and
a client device configured to display the visualization.

2. The repository and analytics system of claim 1, wherein the plurality of data source devices includes a first data source device that is configured to provide a first set of neurological data and a second data source device that is configured to provide a second set of neurological data, wherein the neurological data includes the first set of neurological data and the second set of neurological data.

3. The repository and analytics system of claim 2, wherein the plurality of data source devices includes a third data source device that is configured to provide a third set of neurological data, wherein the first set of neurological data is collected from a first subject and the second set of neurological data is collected from a second subject.

4. The repository and analytics system of claim 2, wherein the first set of neurological data is in a first format and the second set of neurological data is in a second format, wherein the repository and analytics platform is configured to standardize or convert the first set of neurological data and the second set of neurological data into a standard format.

5. The repository and analytics system of claim 2, wherein the repository and analytics platform is configured to:
determine a shared attribute between a subset of the first set of neurological data and a subset of the second set of neurological data; and
combine or link the subset of the first set of neurological data with the subset of the second set of neurological data to form the linked set of neurological data based on the shared attribute.

6. The repository and analytics system of claim 1, wherein the plurality of data source devices includes at least one of an electroencephalogram scanner, a magnetic resonance imager, or a diffusion tensor imager.

7. The repository and analytics system of claim 1, wherein the neurological data includes multi-modal data including neuroimaging, electrophysiology, molecular sample data, serological sample data or tissue sample data.

8. The repository and analytics system of claim 1, wherein the repository and analytics platform is further configured to identify a plurality of biomarkers indicating epileptogenesis.

9. The repository and analytics system of claim 1, wherein the repository and analytics platform is further configured to:
de-identify the neurological data including removing any personal identifiable information;
assign a global unique identifier to the neurological data information; and
validate a quality of the neurological data.

10. A repository and analytics platform, comprising:
a memory; and
one or more processors coupled to the memory and configured to execute instructions store in the memory and perform operations comprising:
obtaining, from a plurality of data source devices, neurological data including a first set of neurological data and a second set of neurological data,
combining or linking a subset of the first set of neurological data with a subset of the second set of neurological data to form a linked set of neurological data,
determining a relationship or pattern within the neurological data based on the linked set of neurological data, and
displaying, on a client device, a visualization that identifies biomarkers of epileptogenesis based on the relationship or pattern.

11. The repository and analytics platform of claim 10, wherein the operations further comprise:
determining a shared attribute between the subset of the first set of neurological data and the subset of the second set of neurological data, wherein combining or linking the subset of the first set of neurological data and the subset of the second set of neurological data is based on the shared attribute.

12. The repository and analytics platform of claim 10, wherein the operations further comprise:
de-identifying the neurological data including removing any personal identifiable information;
assigning a global unique identifier to the neurological data information; and
validating a quality of the neurological data.

13. The repository and analytics platform of claim 10, wherein the plurality of data source devices includes at least one of an electroencephalogram scanner, a magnetic resonance imager, or a diffusion tensor imager.

14. The repository and analytics platform of claim 10, wherein the neurological data includes multi-modal data including neuroimaging, electrophysiology, molecular sample data, serological sample data or tissue sample data.

15. The repository and analytics platform of claim 10, wherein the operations further comprise:
identifying a plurality of biomarkers indicating epileptogenesis, wherein the displaying the visualization is based on the identified plurality of biomarkers.

16. The repository and analytics platform of claim 10, wherein the first set of neurological data is in a first format and the second set of neurological data is in a second format, wherein the operations further comprise:
converting the first set of neurological data and the second set of neurological data into a standard format.

17. A method for identifying biomarkers, comprising:
obtaining, from a plurality of data source devices and using a processor, neurological data including a first set of neurological data and a second set of neurological data;
combining or linking, using the processor, a subset of the first set of neurological data with a subset of the second set of neurological data to form a linked set of neurological data;
determining, using the processor, a relationship or pattern within the neurological data based on the linked set of neurological data; and
displaying, on a client device and using the processor, a visualization that identifies biomarkers of epileptogenesis based on the relationship or pattern.

18. The method of claim 17, further comprising:
converting the first set of neurological data and the second set of neurological data into a standard format.

19. The method of claim 17, further comprising:
identifying a plurality of biomarkers indicating epileptogenesis, wherein the displaying the visualization is further based on the identified plurality of biomarkers.

20. The method of claim 17, further comprising:
de-identifying the neurological data including removing any personal identifiable information;
assigning a global unique identifier to the neurological data information; and
validating a quality of the neurological data.
